(19) United States
(12) Patent Application Publication
(10) Pub. No.: US 2005/0025813 A1
(43) Pub. Date: Feb. 3, 2005

(54) Keratin layer improving laminated sheet

(75) Inventors: Masaru Hoshi, Warabi-shi (JP); Yasunari Takanashi, Warabi-shi (JP); Kaname Nakahara, Warabi-shi (JP); Norikazu Sato, Osaka (JP); Kyoko Hashino, Osaka (JP); Kanae Morishita, Osaka (JP)

Correspondence Address: Kent E. Baldauf, Sr., 700 Koppers Building, 436 Seventh Avenue, Pittsburgh, PA, 15219-1818, US

(73) Assignee: ROHTO PHARMACEUTICAL CO., LTD.

(21) Appl. No.: 10/903,834

(22) Filed: Jul. 30, 2004

(30) Foreign Application Priority Data
Aug. 1, 2003 (JP) 2003-285087

(51) Int. Cl. A61K 9/70

(52) U.S. Cl. 424/443

(57) ABSTRACT

Disclosed is a keratin layer improving laminated sheet comprising a fixing adhesive sheet having an adhesive layer on an adhesive sheet substrate and a moisture retaining pad having a moisturizing agent-containing polymer layer on a pad substrate, said moisture retaining pad being fixed to the fixing adhesive sheet in such a manner that the pad substrate of the moisture retaining pad is in contact with the adhesive layer of the fixing adhesive sheet, wherein in 100% by weight of the moisturizing agent-containing polymer layer, glycerol as a moisturizing agent is contained in an amount of 25 to 55% by weight and moisturizing agents including glycerol are contained in a total amount of 30 to 60% by weight.

### FIELD OF THE INVENTION

The present invention relates to laminated sheet type patches for keratin layer improvement to soften horny layers of the hardened skin of elbows, knees, heels, fingertips or the like and to retain moisture content of the skin.

### BACKGROUND OF THE INVENTION

It has been conventionally known that vitamin E, vitamin A, olive oil, squalane, rice bran, etc. are remedies for skin roughness such as chaps, and lotions, ointments, emulsions and the like containing these ingredients are on the market.

These formulations, however, are liquid or semi-solid, and therefore, fingers are stained when they are applied. Moreover, after application of these formulations, clothes or bedclothes are brought into contact with the applied area to wipe the above ingredients away, so that satisfactory effects cannot be obtained.

In contrast therewith, sheet type patches have been proposed for the purpose of preventing ingredient loss attributable to contact with clothes or bedclothes or solving troublesome application.

For example, there has been proposed a keratin layer improving sheet wherein an adhesive layer containing a keratin layer improving agent is formed on a sheet (see patent document 1). In this document, a remedy for keratinizing skin disease and a moisturizing agent are mentioned as the keratin layer improving agents, and it is described that it is effective to add the moisturizing agents in amounts of 3 to 45% by weight based on the whole adhesive. In case of such a patch, however, even if a satisfactory moisture retaining effect is obtained while the sheet is sticking to the skin, the moisture retaining effect is decreased after the sheet is peeled off from the skin.

There has been further proposed a patch for curing skin roughness wherein an adhesive layer is formed on a substrate similarly to the above and 6 to 30% (w/w) of glycerol is contained in the adhesive layer in expectation of immediate action and prolonged action (see patent document 2). In case of this patch, however, if glycerol is contained in a large amount, it exudes to the surface of the adhesive layer to deteriorate adhesion properties, or cohesive force of the adhesive layer is decreased to bring about cohesive failure, whereby remaining of the adhesive layer on the application area (adhesive transfer) or stickiness is sometimes brought about.

Patent document 1: Japanese Patent Laid-Open Publication No. 147820/1999

Patent document 2: Japanese Patent Laid-Open Publication No. 281565/2000

### OBJECT OF THE INVENTION

It is an object of the present invention to provide a keratin layer improving laminated sheet capable of containing moisturizing agents such as glycerol in large amounts, capable of diffusing the moisturizing agents into the applied area of the skin when the sheet is applied to the skin and capable of maintaining a moisture retaining effect on the skin not only while the sheet is sticking to the skin but also after the sheet is peeled off from the skin.

### SUMMARY OF THE INVENTION

The present inventors have earnestly studied to solve the above problems, and as a result, they have found that in a patch of laminated type comprising a fixing adhesive sheet and a moisture retaining pad, large amounts of moisturizing agents such as glycerol can be contained in the moisture retaining pad, and they have also found that by application of the patch to the skin, a moisture retaining effect can be maintained not only while the patch is sticking to the skin but also after the patch is peeled off from the skin. Based on the finding, the present invention has been accomplished.

That is to say, the keratin layer improving laminated sheet according to the present invention is a keratin layer improving laminated sheet comprising a fixing adhesive sheet having an adhesive layer on an adhesive sheet substrate and a moisture retaining pad having a moisturizing agent-containing polymer layer on a pad substrate, said moisture retaining pad being fixed to the fixing adhesive sheet in such a manner that the pad substrate of the moisture retaining pad is in contact with the adhesive layer of the fixing adhesive sheet, wherein in 100% by weight of the moisturizing agent-containing polymer layer, glycerol as a moisturizing agent is contained in an amount of 25 to 55% by weight and moisturizing agents including glycerol are contained in a total amount of 30 to 60% by weight.

It is preferable that urea is contained as the moisturizing agent in addition to glycerol.

It is also preferable that a water-soluble moisturizing agent and an oil-soluble moisturizing agent are contained as the moisturizing agents.

In the present invention, further, it is preferable that in 100% by weight of the moisturizing agent-containing polymer layer, a partially crosslinked binder polymer is contained in an amount of 35 to 60% by weight.

The moisture retaining pad preferably has an adhesive force, as measured in accordance with JIS Z 0237, of not more than.0.5 N/19 mm.

The moisture retaining pad preferably has a ball tack value, as measured in accordance with JIS Z 0237, of not more than 4.

The fixing adhesive sheet preferably has an adhesive force, as measured in accordance with JIS Z 0237, of 2 to 12 N/19 mm.

### BRIEF DESCRIPTION OF THE DRAWING

FIG. 1 is a schematic view of a keratin layer improving laminated sheet according to the present invention.

### DETAILED DESCRIPTION OF THE INVENTION

The present invention is described in detail hereinafter.

The keratin layer improving laminated sheet of the invention is a keratin layer improving laminated sheet comprising a fixing adhesive sheet having an adhesive layer on an adhesive sheet substrate and a moisture retaining pad having a moisturizing agent-containing polymer layer on a pad substrate, said moisture retaining pad being fixed to the fixing adhesive sheet in such a manner that the pad substrate of the moisture retaining pad is in contact with the adhesive layer of the fixing adhesive sheet, wherein in 100% by weight of the moisturizing agent-containing polymer layer, glycerol as a moisturizing agent is contained in an amount of 25 to 55% by weight and moisturizing agents including glycerol are contained in a total amount of 30 to 60% by weight.

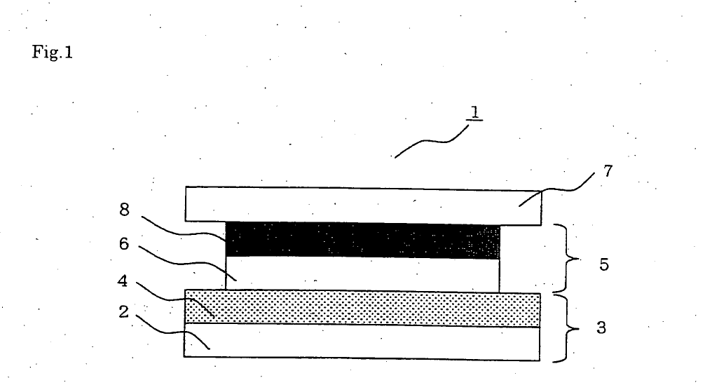

First, the structure of the keratin layer improving laminated sheet of the invention is described with reference to FIG. 1.

In FIG. 1, a keratin layer improving laminated sheet 1 comprises a fixing adhesive sheet 3 and a moisture retaining pad 5, and this laminated sheet 1 may further have a release member 7 when needed. By virtue of such a structure, the keratin layer improving laminated sheet 1 can be applied and fixed to the skin with the fixing adhesive sheet 3 even if the adhesive force and the ball tack value of the moisture retaining pad 5 are low When the release member 7 is provided in the laminated sheet, the unused moisture retaining pad 5 and fixing adhesive sheet 3 can be protected from drying or adhesion of dust.

The fixing adhesive sheet 3 comprises an adhesive sheet substrate 2 and an adhesive layer 4 formed thereon, and the moisture retaining pad 5 comprises a pad substrate 6 and a moisturizing agent-containing polymer layer 8 formed thereon.

To the fixing adhesive sheet 3, the moisture retaining pad 5 is fixed, and in this case, the moisture retaining pad 5 is fixed to the fixing adhesive sheet 3 in such a manner that the pad substrate 6 of the moisture retaining pad 5 is in contact with the adhesive layer 4 of the fixing adhesive sheet 3.

The sizes of the fixing adhesive sheet 3 and the moisture retaining pad 5 are not specifically restricted and can be each determined according to necessity. However, the size of the fixing adhesive sheet 3 is preferably larger than the size of the moisture retaining pad 5 from the viewpoint that the fixing adhesive sheet 3 functions to fix the moisture retaining pad 5 to form the keratin layer improving laminated sheet 1 and functions to stick and fix the laminated sheet to the skin. More specifically, in the case where the fixing adhesive sheet 3 and the moisture retaining pad 5 are in the rectangular forms congruent with each other, it is preferable that when the moisture retaining pad 5 is laminated on the fixing adhesive sheet 3 and fixed thereto, portions of the fixing adhesive sheet 3, which are not used for the lamination between the moisture retaining pad 5 and the fixing adhesive sheet 3, are present in a width of not less than 5 mm based on one side.

Next, the constituents are described in detail.

As the adhesive sheet substrate 2 to constitute the fixing adhesive sheet 3, materials having follow-up properties to the skin, namely, flexibility can be employed without any restriction. More specifically, there can be mentioned, for example, resin films, non-woven fabrics, fabrics, and laminates thereof. As the resin films, films of publicly known polymers are employable, and examples of the polymers include polyethylene, polypropylene, polyvinyl chloride, polyurethane, polyester and an ethylene/vinyl acetate copolymer. The non-woven fabrics and the fabrics are not specifically restricted, and they can be obtained by the use of synthetic fibers, such as polyethylene, polypropylene, polyurethane and polyester, or natural fibers, such as cotton and silk. When the substrate 2 is a non-woven fabric or a fabric, one kind of the above fibers may be used for the fabric, or a mixture of two or more kinds of the above fibers may be used for the fabric.

As the adhesive used for the adhesive layer 4 to constitute the fixing adhesive sheet 3, an adhesive that is less irritant to the skin is preferably employed, and there can be mentioned solvent type adhesives, emulsion type adhesives and hot melt type adhesives, without limiting thereto. More specifically, there can be mentioned, for example, adhesives containing a homopolymer or a copolymer of (meth)acrylic acid, a homopolymer or a copolymer of (meth)acrylic ester, a copolymer of (meth)acrylic acid and (meth)acrylic ester, a urethane polymer, a rubber type polymer or a silicon type polymer. The adhesive may contain a crosslinking agent publicly known.

The fixing adhesive sheet 3 has an adhesive force, as measured in accordance with JIS Z 0237, of usually 2 to 12 N/19 mm, preferably 3 to 10 N/19 mm. When the adhesive force of the fixing adhesive sheet 3 is in the above range, the keratin layer improving laminated sheet 1 can be fixed to the skin for an enough time to exert the effect of the present invention.

The pad substrate 6 to constitute the moisture retaining pad 5 is preferably one having good adhesion to a partially crosslinked binder polymer used for the later-described moisturizing agent-containing polymer layer 8, and examples of such materials include those previously described for the adhesive sheet substrate 2, such as resin films, non-woven fabrics, fabrics and laminates thereof, without limiting thereto.

Of such materials, preferable are non-woven fabrics and fabrics obtained by the use of synthetic fibers, such as polyethylene, polypropylene, polyurethane and polyester, or natural fibers, such as cotton and silk. For such fabrics, one kind of the above fibers may be used, or a mixture of two or more kinds of the above fibers may be used.

In order to retain the moisture content of the skin in the applied area and to allow the moisture retention properties of the moisture retaining pad 5 to exhibit sufficiently, it is preferable to use a laminate of the non-woven fabric or the fabric and a moisture-impermeable film (e.g., moisture-impermeable films selected from the resin films previously exemplified for the adhesive sheet substrate 2).

The moisturizing agent-containing polymer layer 8 to constitute the moisture retaining pad 5 contains glycerol as a moisturizing agent in an amount of 25 to 55% by weight and contains moisturizing agents including glycerol in a total amount of 30 to 60% by weight, in 100% by weight of the moisturizing agent-containing polymer layer 8.

Examples of the moisturizing agents include:

water-soluble moisturizing agents, specifically, polyhydric alcohols, such as glycerol, 1,3-butylene glycol, polyethylene glycol, sorbitol and maltitol, carboxylic acids or salts thereof, such as hyaluronic acid, sodium hyaluronate, dl-pyridonecarboxylic acid and dipotassium glycyrrhetinate, saccharides, such as grape sugar and fruit sugar, amino acids, such as trimethylglycine, extracts obtained by extraction with water, such as Shiso extract, aloe extract, hypha extract, placenta extract, mugwort extract, licorice extract, Arunica extract, marronnier (horse chestnut tree) extract and yeast extract, hydroxyproline, betain, and urea; and

oil-soluble moisturizing agents, specifically, extracts from animals and vegetables, such as sunflower oil, jojoba oil, olive oil, peanut oil, soybean oil, tsubaki oil, Bayu (horse fat oil), lanolin, rosemary, common lime and soybean phospholipid, cetanol, bio-γ-linolenin, lecithin, ceramide, cholesterol, squalane, vaseline, fatty acids, and fatty esters.

In the present specification, the “water-soluble moisturizing agents” mean water-soluble substances selected from substances capable of retaining moisture content of the skin to contribute moisture retention. The “oil-soluble moisturizing agents” mean oil-soluble substances selected from substances capable of retaining moisture content of the skin to contribute moisture retention, and they are intended to include so-called oils, such as animal and vegetable oils. These animal and vegetable oils can retain moisture content of the skin by covering the surface of the skin with an oil film.

Of the above substances, glycerol is contained as an essential moisturizing agent in the present invention. That is to say, in 100% by weight of the moisturizing agent-containing polymer layer 8, glycerol is contained in an amount of usually 25 to 55% by weight, preferably 30 to 55% by weight, more preferably 35 to 55% by weight, still more preferably 40 to 55% by weight. When glycerol is contained in an amount of the above range, the moisture retaining effect on the skin is enhanced, and even after the keratin layer improving laminated sheet 1 is peeled off from the skin, the moisture retaining effect can be maintained over a long period of time.

In the present invention, further, it is desiable that moisturizing agents including glycerol are contained in a total amount of usually 30 to 60% by weight, preferably 40 to 60% by weight, more preferably 45 to 60% by weight, still more preferably 50 to 60% by weight, in 100% by weight of the moisturizing agent-containing polymer layer 8.

That is to say, in addition to glycerol, a moisture retaining ingredient other than glycerol is preferably contained as the moisturizing agent. Examples of the moisturizing agents other than glycerol include those previously described as the moisturizing agents (except glycerol). Of those, urea is preferable as the water-soluble moisturizing agent, and a vegetable oil such as sunflower oil or jojoba oil is preferable as the oil-soluble moisturizing agent.

In the present invention, it is more desirable that the water-soluble moisturizing agent and the oil-soluble moisturizing agent are used in combination. When the water-soluble moisturizing agent and the oil-soluble moisturizing agent are used in combination, the oil-soluble moisturizing agent is desirably contained in an amount of usually 5 to 40% by weight in 100% by weight of the moisturizing agents.

In the present invention, further, it is preferable that in 100% by weight of the moisturizing agent-containing polymer layer 8, a partially crosslinked binder polymer is contained in an amount of usually 35 to 60% by weight.

The partially crosslinked binder polymer is a polymer obtained by partially crosslinking a partially crosslinkable polymer through physical crosslinking or chemical crosslinking and has high holding power, so that the moisturizing agents can be contained in large amounts.

More specifically, even if moisturizing agents including glycerol are contained in the binder polymer in large amounts, exudation of the moisturizing agents or wetting with said agents does not occur. Moreover, cohesive failure does not occur either, and hence, even after the keratin layer improving laminated sheet is peeled off from the skin, neither adhesive transfer nor stickiness is brought about.

The partially crosslinked binder polymer has a holding power, as measured in accordance with JIS Z 0237, of usually not less than 10000 seconds, preferably not less than 30000 seconds, more preferably not less than 50000 seconds.

The binder polymer is preferably a polymer capable of holding both of a water-soluble base material and an oil-soluble base material from the viewpoint that not only a water-soluble moisturizing agent but also an oil-soluble moisturizing agent can be favorably contained in the polymer.

Examples of the partially crosslinkable polymers used for forming the partially crosslinked binder polymers include a homopolymer or a copolymer of (meth)acrylic acid, a homopolymer or a copolymer of (meth)acrylic ester, a copolymer of (meth)acrylic acid and (meth)acrylic ester, and a rubber type polymer. Of these, a copolymer of (meth)acrylic acid and (meth)acrylic ester is preferable.

Examples of crosslinking agents to chemically crosslink the partially crosslinkable polymers include aziridine type crosslinking agents, such as hexamethylenediethyleneurea, isocyanate type crosslinking agents, such as polyisocyanate, epoxy type crosslinking agents, melamine type crosslinking agents and carboxylic acid type crosslinking agents. In case of chemical crossinking, the crosslinking agent is used in an amount of usually 0.01 to 2 parts by weight, preferably 0.05 to 1.5 parts by weight, based on 100 parts by weight of the partially crosslinkable polymer, to crosslink the partially crosslinkable polymer, whereby a partially crosslinked binder polymer can be obtained.

In the present invention, the partially crosslinked binder polymer is contained in an amount of usually 35 to 60% by weight, preferably 35 to 55% by weight, more preferably 40 to 55% by weight, in 100% by weight of the moisturizing agent-containing polymer layer 8.

When the partially crosslinked binder polymer is contained in an amount of the above range, the amounts of the moisturizing agents in the moisturizing agent-containing polymer layer 8 can be relatively increased. Further, the binder polymer has a high holding power, as previously described, and therefore, even if the moisturizing agents are contained in large amounts, exudation of the moisturizing agents or wetting with said agents does not occur. Moreover, cohesive failure does not occur either, and hence, even after the keratin layer improving laminated sheet is peeled off from the skin, neither adhesive transfer nor stickiness is brought about.

The moisturizing agent-containing polymer layer 8 may further contain other components, for example, stabilizers, such as tocopherol, paraben and dextrin palmitate, keratin layer softeners, such as fruit acid, vitamins, colorants used for the purpose of discriminating users, perfumes, surface active agents, pH adjustors and water, within limits not detrimental to the object of the present invention. These other components may be contained in a total amount of usually 0 to 10% by weight in 100% by weight of the moisturizing agent-containing polymer layer 8.

The moisture retaining pad 5 has an adhesive force, as measured in accordance with JIS Z 0237, of usually not more than 0.5 N/19 mm, and this adhesive force may be in the range of 0 to 0.3 N/19 mm. The moisture retaining pad 5 has a ball tack value, as measured in accordance with JIS Z 0237, of usually not more than 4, and this ball tack value may be in the range of 0 to 3.

Although the moisture retaining pad 5 has an adhesive force of not more than 0.5 N/19 mm and a ball tack value of not more than 4, the moisture retaining pad 5 can be sufficiently stuck and fixed to the skin because the moisture retaining pad 5 forms a laminated structure together with the fixing adhesive sheet 3 in the present invention. That is to say, even if the moisturizing agents including glycerol are contained in the moisture retaining pad 5 in large amounts, the keratin layer improving laminated sheet 1 can be fixed to the skin for an enough time to exert the effect of the present invention because the laminated sheet 1 comprises the moisture retaining pad 5 and the fixing adhesive sheet 3.

In the keratin layer improving laminated sheet 1 of the invention, a release member 7 may be provided to protect the moisturizing agent-containing polymer layer 8 and the adhesive layer 4 of the fixing adhesive sheet 3 from drying and adhesion of dust.

The release member 7 has only to be a member in which one or both surfaces of a paper or a resin film are coated with a release agent such as a silicone resin, and publicly known materials can be used without any restriction. However, preferable is a material whose peel strength from the moisturizing agent-containing polymer layer 8 and the adhesive layer 4 of the fixing adhesive sheet 3 is not so high.

### EFFECT OF THE INVENTION

According to the keratin layer improving laminated sheet of the invention, moisturizing agents such as glycerol can be contained in the moisture retaining pad in large amounts without exudation of the moisturizing agents to the surface of the moisture retaining pad or wetting of the fixing adhesive sheet with said agents, and by application of the sheet to the skin, the moisturizing agents are effectively diffused into the applied area of the skin and the moisture retaining effect can be maintained not only while the laminated sheet is sticking to the skin but also after the laminated sheet is peeled off from the skin.

In the keratin layer improving laminated sheet of the invention, the adhesive force and the ball tack value of the moisture retaining pad are low, but they can be compensated by virtue of the laminated structure comprising the moisture retaining pad and the fixing adhesive sheet. Further, a partially crosslinked binder polymer is contained in the moisture retaining pad, and therefore, even if large amounts of moisturizing agents are contained in the moisture retaining pad, cohesive failure does not take place. Hence, there is brought about neither adhesive transfer nor stickiness, and excellent use feeling is given.

In the keratin layer improving laminated sheet of the invention, moreover, a water-soluble moisturizing agent and an oil-soluble moisturizing agent are used in combination, whereby the surface of the skin can be covered with an oil film of the oil-soluble moisturizing agent to inhibit moisture loss from the skin surface, and besides, permeation of the water-soluble moisturizing agent into the horny layer can be accelerated.

### EXAMPLE

The present invention is further described with reference to the following examples, but it should be construed that the invention is in no way limited to those examples.

### Example 1

### Preparation of Fixing Adhesive Sheet

To an ethyl acetate solution of a 2-ethylhexyl acrylate/butyl acrylate/methacrylic acid (60/35/5, % by weight) copolymer, polyisocyanate was added as a crosslinking agent in an amount of 1.5 parts by weight based on 100 parts by weight of solid content of the copolymer, and they were homogenously stirred.

Then, the solution was applied onto a release member made of a polyester film (thickness: 38 μm) having been treated with a silicone resin, in such a manner that a uniform layer (thickness after dried: 25 μm) was obtained, and the coating was heated by a heater to remove the solvent, whereby an adhesive layer was formed on the release member.

The adhesive layer was laminated onto an adhesive sheet substrate made of a polyester non-woven fabric (weight: 50 g/m²), and the resulting laminate was cut into a rectangle of 130 mm×90 mm to obtain a fixing adhesive sheet with a release member.

### Preparation of Moisture Retaining Pad

A moisture retaining pad was prepared in accordance with the composition and the compounding ratio shown in Table 1.

In more detail, a solution A obtained by mixing jojoba oil with sunflower oil was mixed with a solution B obtained by adding urea and fruit acid to glycerol, and the mixture of the solution A and the solution B and hexamethylenediethyleneurea were added to an ethyl acetate solution of a 2-ethylhexyl acrylate/butyl acrylate/methacrylic acid (58/37/5, % by weight) copolymer, followed by stirring for 20 minutes.

The resulting solution was applied onto a release member made of a polyester film (thickness: 38 μm) having been treated with a silicone resin, in such a manner that a uniform layer (thickness after dried: 60 μm) was obtained, and the coating was heated by a heater to remove the solvent, whereby a moisturizing agent-containing polymer layer was formed on the release member.

The moisturizing agent-containing polymer layer was laminated onto a pad substrate made of a laminate of a polyester non-woven fabric (weight: 50 g/m²) and a polyethylene film (thickness: 30 μm) in such a manner that the polymer layer was in contact with the polyester non-woven fabric, and the resulting laminate was cut into a rectangle of 100 mm×60 mm to obtain a moisture retaining pad with a release member.

### Preparation of Keratin Layer Improving Laminated Sheet

From the fixing adhesive sheet with a release member obtained as above, the release member was peeled off, and the fixing adhesive sheet was put so that the adhesive layer should be on the upper side. Then, from the moisture retaining pad with a release member, the release member was peeled off, and the moisture retaining pad was placed on the prescribed area of the fixing adhesive sheet in such a manner that the pad substrate was in contact with the adhesive layer of the fixing adhesive sheet to give a laminate.

On the laminate, a release member made of a polyester film (thickness: 50 μm) having been treated with a silicone resin was further laminated. Thus, a keratin layer improving laminated sheet with a release member was obtained.

### Example 2

A keratin layer improving laminated sheet with a release member was obtained in the same manner as in Example 1, except that the composition and the compounding ratio of the moisture retaining pad were changed as shown in Table 1.

### Example 3

A keratin layer improving laminated sheet with a release member was obtained in the same manner as in Example 1, except that in the preparation of moisture retaining pad, a solution obtained by mixing jojoba oil with squalane was used as the solution A and the composition and the compounding ratio of the moisture retaining pad were changed as shown in Table 1.

### Example 4

A keratin layer improving laminated sheet with a release member was obtained in the same manner as in Example 1, except that in the preparation of moisture retaining pad, a solution obtained by mixing sunflower oil with squalane was used as the solution A and the composition and the compounding ratio of the moisture retaining pad were changed as shown in Table 1.

### Example 5

A keratin layer improving laminated sheet with a release member was obtained in the same manner as in Example 1, except that the composition and the compounding ratio of the moisture retaining pad were changed as shown in Table 1.

### Example 6

A keratin layer improving laminated sheet with a release member was obtained in the same manner as in Example 1, except that in the preparation of moisture retaining pad, jojoba oil was used as the solution A and the composition and the compounding ratio of the moisture retaining pad were changed as shown in Table 1.

### Comparative Example 1

A keratin layer improving laminated sheet with a release member was obtained in the same manner as in Example 1, except that in the preparation of moisture retaining pad, the solution A was not used, a solution obtained by adding urea to glycerol was used as the solution B, the solution B and hexamethylenediethyleneurea were added to an ethyl acetate solution of a 2-ethylhexyl acrylate/butyl acrylate/methacrylic acid (58/37/5, % by weight) copolymer, and the composition and the compounding ratio of the moisture retaining pad were changed as shown in Table 1.

### Comparative Example 2

A keratin layer improving laminated sheet with a release member was obtained in the same manner as in Example 1, except that the composition and the compounding ratio of the moisture retaining pad were changed as shown in Table 1.

### Comparative Example 3

A keratin layer improving laminated sheet with a release member was obtained in the same manner as in Example 1, except that in the preparation of moisture retaining pad, the solution A was not used, glycerol was used as the solution B, the solution B and hexamethylenediethyleneurea were added to an ethyl acetate solution of a 2-ethylhexyl acrylate/butyl acrylate/methacrylic acid (58/37/5, % by weight) copolymer, and the composition and the compounding ratio of the moisture retaining pad were changed as shown in Table 1.

### Comparative Example 4

A fixing adhesive sheet was prepared in the same manner as in Example 1, except that the compounding ratio of the fixing adhesive sheet was changed as shown in Table 1, and a moisture retaining pad was prepared in the same manner as in Comparative Example 3. Using the fixing adhesive sheet and the moisture retaining pad, a keratin layer improving laminated sheet with a release member was obtained in the same manner as in Example 1.

### Test Example

The partially crosslinked binder polymer, the moisture retaining pad, the fixing adhesive sheet and the keratin layer improving laminated sheet were evaluated in accordance with the following evaluation methods.

(1) Evaluation of Holding Power of Partially Crosslinked Binder Polymer

Pads composed of partially crosslinked binder polymers were prepared in the same manner as in the above-described preparation of moisture retaining pad, except that the moisturizing agents (glycerol, urea, jojoba oil, sunflower oil, squalane) and the keratin layer softening agent (fruit acid) were not used, but hexamethylenediethyleneurea and an ethyl acetate solution of a 2-ethylhexyl acrylate/butyl acrylate/methacrylic acid (58/37/5, % by weight) copolymer were used in the compounding ratios of Examples 1 to 6 and Comparative Examples 1 to 4 shown in Table 1.

The holding power of each of the resulting pads was measured in accordance with JIS Z 0237.

The results are set forth in Table 1.

(2) Evaluation of Adhesive Force of Moisture Retaining Pad

The moisture retaining pad with a release member prepared in each of Examples 1 to 6 and Comparative Examples 1 to 4 was cut in a width of 19 mm, and the release member was peeled off to obtain a specimen. The adhesive force of, each specimen was measured in accordance with JIS Z 0237.

The results are set forth in Table 1.

(3) Ball Tack Value of Moisture Retaining Pad

From the moisture retaining pad with a release member prepared in each of Examples 1 to 6 and Comparative Examples 1 to 4, the release member was peeled off, and the ball tack value was measured in accordance with JIS Z 0237.

The results are set forth in Table 1.

(4) Evaluation of Adhesive Force of Fixing Adhesive Sheet

The fixing adhesive sheet with a release member prepared in each of Examples 1 to 6 and Comparative Examples 1 to 4 was cut in a width of 19 mm, and the release member was peeled off to obtain a specimen. The adhesive force of each specimen was measured in accordance with JIS Z 0237.

The results are set forth in Table 1.

(5) Evaluation of Keratin Layer Improving Effect

From the keratin layer improving laminated sheet with a release member prepared in each of Examples 1 to 6 and Comparative Examples 1 to 4, the release member was peeled off. Then, each laminated sheet was applied to an applicable region (heel) of 10 monitors, and after 8 hours from the application, the laminated sheet was peeled off. Immediately after the peeling and after 4 hours from the peeling, the moisture retaining effect (feeling of moisture retention) was evaluated based on the following criteria.

The results are set forth in Table 1. Evaluation points in Table 1 are average values of 10 monitors.

5 points: There is satisfactory feeling of moisture retention.

4 points: There is moderate feeling of moisture retention.

3 points: There is a little feeling of moisture retention.

2 points: There is almost no feeling of moisture retention.

1 point: There is no feeling of moisture retention.

(6) Evaluation of Stickiness and Adhesive Transfer

From the keratin layer improving laminated sheet with a release-member prepared in each of Examples 1 to 6 and Comparative Examples 1 to 4, the release member was peeled off. Then, each laminated sheet was applied to an applicable region (heel) of 10 monitors, and after 8 hours from the application, the laminated sheet was peeled off. Immediately after the peeling, feeling of stickiness was evaluated based on the following criteria.

The results are set forth in Table 1. Evaluation points in Table 1 are average values of 10 monitors.

Adhesive transfer was not observed in any of the examples.

5 points: There is no stickiness.

4 points: There is slight stickiness.

3 points: There is a little stickiness.

2 points: There is considerable stickiness.

1 point: There is marked stickiness.

TABLE 1

| | | | | | | | | | Comp. | Comp. | Comp. | Comp. |
|---|---|---|---|---|---|---|---|---|---|---|---|---|
| | | | Ex. 1 | Ex. 2 | Ex. 3 | Ex. 4 | Ex. 5 | EX. 6 | Ex. 1 | Ex. 2 | Ex. 3 | Ex. 4 |
| Fixing | Acrylic ester | (part(s) by | 100 | 100 | 100 | 100 | 100 | 100 | 100 | 100 | 100 | 100 |
| adhesive | copolymer 1* | weight) | | | | | | | | | | |
| sheet | Polyisocyanate | (part(s) by | 1.5 | 1.5 | 1.5 | 1.5 | 1.5 | 1.5 | 1.5 | 1.5 | 1.5 | 6 |
| | | weight) | | | | | | | | | | |
| Moisture | Acrylic ester | (% by weight) | 44.6 | 54.6 | 44.6 | 44.6 | 40.6 | 41.6 | 81.6 | 71.6 | 24.6 | 24.6 |
| retaining | copolymer 2* | | | | | | | | | | | |
| pad | Hexamethylene- | (% by weight) | 0.4 | 0.4 | 0.4 | 0.4 | 0.4 | 0.4 | 0.4 | 0.4 | 0.4 | 0.4 |
| | diethyleneurea | | | | | | | | | | | |
| | Glycerol | (% by weight) | 30.0 | 30.0 | 30.0 | 30.0 | 40.0 | 50.0 | 15.0 | 15.0 | 75.0 | 75.0 |
| | Urea | (% by weight) | 3.0 | 3.0 | 3.0 | 3.0 | 3.0 | 3.0 | 3.0 | 3.0 | — | — |
| | Jojoba oil | (% by weight) | 10.0 | 5.0 | 10.0 | — | 7.5 | 3.0 | — | 5.0 | — | — |
| | Sunflower oil | (% by weight) | 10.0 | 5.0 | — | 10.0 | 7.5 | — | — | 5.0 | — | — |
| | Squalane | (% by weight) | — | — | 10.0 | 10.0 | — | — | — | — | — | — |
| | Fruit acid | (% by weight) | 2.0 | 2.0 | 2.0 | 2.0 | 1.0 | 2.0 | — | — | — | — |
| | Content of | (% by weight) | 53.0 | 43.0 | 53.0 | 53.0 | 58.0 | 56.0 | 18.0 | 28.0 | 75.0 | 75.0 |
| | moisturizing | | | | | | | | | | | |
| | agent | | | | | | | | | | | |
| Evaluation | (1) Holding power | (second(s)) | 70000 | 70000 | 70000 | 70000 | 70000 | 70000 | 70000 | 70000 | 70000 | 70000NC |
| results | of binder polymer | | NC | NC | NC | NC | NC | NC | NC | NC | NC | NC |
| | (2) Adhesive force | (N/19 mm) | 0.1 or | 0.1 or | 0.1 or | 0.1 or | 0.1 or | 0.1 or | 8.3 | 5.5 | 0.1 or | 0.1 or |
| | of moisture retain- | less | less | less | less | less | less | | | less | less | |
| | ing pad | | | | | | | | | | | |
| | (3) Ball tack value | (ball No.) | 1 or less | 1 or less | 1 or less | 1 or less | 1 or less | 1 or less | 12 | 6 | 1 or less | 1 or less |
| | of moisture retain- | | | | | | | | | | | |
| | ing pad | | | | | | | | | | | |
| | (4) Adhesive force | (N/19 mm) | 8.8 | 8.8 | 8.8 | 8.8 | 8.8 | 8.8 | 8.8 | 8.8 | 8.8 | 1.6 |
| | of fixing adhesive | | | | | | | | | | | |
| | sheet | | | | | | | | | | | |
| | (5-1) Feeling of | (point(s)) | 5 | 4 | 5 | 5 | 4 | 5 | 2 | 3 | 3 | — |
| | moisture retention | | | | | | | | | | | |
| | immediately after | | | | | | | | | | | |
| | peeling | | | | | | | | | | | |
| | (5-2) Feeling of | (point(s)) | 4 | 4 | 4 | 5 | 4 | 4 | 1 | 1 | 1 | — |
| | moisture retention | | | | | | | | | | | |
| | after 4 hours from | | | | | | | | | | | |
| | peeling | | | | | | | | | | | |
| | (6) Stickiness | (point(s)) | 5 | 5 | 5 | 5 | 5 | 5 | 5 | 5 | 1 | — |

*Acrylic ester copolymer 1: 2-ethylhexyl acrylate/butyl acrylate/methacrylic acid (60/35/5) copolymer
*Acrylic ester copolymer 2: 2-ethylhexyl acrylate/butyl acrylate/methacrylic acid (58/37/5) copolymer
*70000NC means that shearing did not occur after the lapse of 70000 seconds.
*In Comparative Example 4, the sheet peeled off from the skin and could not be sticking to the skin for a given period of time in the evaluation test, and hence, the evaluations (5-1) to (6) could not be carried out.

1. A keratin layer improving laminated sheet comprising a fixing adhesive sheet having an adhesive layer on an adhesive sheet substrate and a moisture retaining pad having a moisturizing agent-containing polymer layer on a pad substrate, said moisture retaining pad being fixed to the fixing adhesive sheet in such a manner that the pad substrate of the moisture retaining pad is in contact with the adhesive layer of the fixing adhesive sheet, wherein in 100% by weight of the moisturizing agent-containing polymer layer, glycerol as a moisturizing agent is contained in an amount of 25 to 55% by weight and moisturizing agents including glycerol are contained in a total amount of 30 to 60% by weight.

2. The keratin layer improving laminated sheet as claimed in claim 1, wherein urea is contained as the moisturizing agent in addition to glycerol.

3. The keratin layer improving laminated sheet as claimed in claim 1, wherein a water-soluble moisturizing agent and an oil-soluble moisturizing agent are contained as the moisturizing agents.

4. The keratin layer improving laminated sheet as claimed in claim 1, wherein in 100% by weight of the moisturizing agent-containing polymer layer, a partially crosslinked binder polymer is contained in an amount of 35 to 60% by weight.

5. The keratin layer improving laminated sheet as claimed in claim 1, wherein the moisture retaining pad has an adhesive force, as measured in accordance with JIS Z 0237, of not more than 0.5 N/19 mm.

6. The keratin layer improving laminated sheet as claimed in claim 1, wherein the moisture retaining pad has a ball tack value, as measured in accordance with JIS Z 0237, of not more than 4.

7. The keratin layer improving laminated sheet as claimed in claim 1, wherein the fixing adhesive sheet has an adhesive force, as measured in accordance with JIS Z 0237, of 2 to 12 N/19 mm.

8. The keratin layer improving laminated sheet as claimed in claim 2, wherein a water-soluble moisturizing agent and an oil-soluble moisturizing agent are contained as the moisturizing agents.

9. The keratin layer improving laminated sheet as claimed in claim 2, wherein in 100% by weight of the moisturizing agent-containing polymer layer, a partially crosslinked binder polymer is contained in an amount of 35 to 60% by weight.

10. The keratin layer improving laminated sheet as claimed in claim 2, wherein the moisture retaining pad has an adhesive force, as measured in accordance with JIS Z 0237, of not more than 0.5 N/19 mm.

11. The keratin layer improving laminated sheet as claimed in claim 2, wherein the moisture retaining pad has a ball tack value, as measured in accordance with JIS Z 0237, of not more than 4.

12. The keratin layer improving laminated sheet as claimed in claim 2, wherein the fixing adhesive sheet has an adhesive force, as measured in accordance with JIS Z 0237, of 2 to 12 N/19 mm.

13. The keratin layer improving laminated sheet as claimed in claim 3, wherein in 100% by weight of the moisturizing agent-containing polymer layer, a partially crosslinked binder polymer is contained in an amount of 35 to 60% by weight.

14. The keratin layer improving laminated sheet as claimed in claim 3, wherein the moisture retaining pad has an adhesive force, as measured in accordance with JIS Z 0237, of not more than 0.5 N/19 mm.

15. The keratin layer improving laminated sheet as claimed in claim 3, wherein the moisture retaining pad has a ball tack value, as measured in accordance with JIS Z 0237, of not more than 4.

16. The keratin layer improving laminated sheet as claimed in claim 3, wherein the fixing adhesive sheet has an adhesive force, as measured in accordance with JIS Z 0237, of 2 to 12 N/19 mm.

17. The keratin layer improving laminated sheet as claimed in claim 4, wherein the moisture retaining pad has an adhesive force, as measured in accordance with JIS Z 0237, of not more than 0.5 N/19 mm.

18. The keratin layer improving laminated sheet as claimed in claim 4, wherein the fixing adhesive sheet has an adhesive force, as measured in accordance with JIS Z 0237, of 2 to 12 N/19 mm.

19. The keratin layer improving laminated sheet as claimed in claim 5, wherein the fixing adhesive sheet has an adhesive force, as measured in accordance with JIS Z 0237, of 2 to 12 N/19 mm.

20. The keratin layer improving laminated sheet as claimed in claim 6, wherein the fixing adhesive sheet has an adhesive force, as measured in accordance with JIS Z 0237, of 2 to 12 N/19 mm.
